(19) United States
(12) Patent Application Publication
(10) Pub. No.: US 2025/0319133 A1
(43) Pub. Date: Oct. 16, 2025

(54) METHODS FOR IMPROVING BONE HEALTH WITH BOVINE MILK EXOSOME-ENRICHED PRODUCTS AND VITAMIN K2

(71) Applicant: ABBOTT LABORATORIES, Abbott Park, IL (US)

(72) Inventors: Ricardo Rueda Cabrera, Granada (ES); José Maria López Pedrosa, Cenes de la Vega (ES); Jorge García Martínez, Armilla (ES)

(21) Appl. No.: 18/866,692

(22) PCT Filed: Nov. 29, 2023
(86) PCT No.: PCT/US2023/081495

§ 371 Date: Nov. 18, 2024

(30) Foreign Application Priority Data
Dec. 5, 2022 (EP) 22383182.7

(51) Int. Cl. A61K 35/20; A61K 9/50; A61K 31/122; A61K 47/46; A61P 19/00; A61P 19/10

(52) U.S. Cl. CPC A61K 35/20; A61K 9/5068; A61K 31/122; A61K 47/46; A61P 19/00; A61P 19/10

(57) ABSTRACT

A method for improving bone formation in an individual comprises administering a bovine milk exosome-enriched product and vitamin K2 to the individual. A method for reducing a risk of bone fracture or strengthening bone in an individual comprises administering a bovine milk exosome-enriched product and vitamin K2 to the individual. A method for preventing or delaying onset or development of osteoporosis in an individual comprises administering a bovine milk exosome-enriched product and vitamin K2 to the individual.

### FIELD OF THE INVENTION

The present invention relates to the use of a bovine milk exosome-enriched product and vitamin K2 for improving bone formation in an individual and/or for reducing a risk of bone fracture or strengthening bone in an individual, as well as the use of a bovine milk exosome-enriched product and vitamin K2 for preventing or delaying onset or development of osteoporosis in an individual.

### BACKGROUND OF THE INVENTION

Bone is a tissue in which the extracellular matrix has been hardened to accommodate a supporting function. Bone consists of approximately 30% water with the remainder composed of various minerals (such as calcium salts), and various cell types including osteoprogenitor cells, osteoblasts, osteocytes, bone lining cells and osteoclasts. Bone development is a lifelong process that involves the integration of multiple signaling pathways and requires coordinated action of these cells.

Bone protects organs from mechanical forces, transmits forces between different areas of the body, and anchors skeletal muscles. Bone is a living, metabolically active tissue that serves as a storehouse for calcium, phosphorous and carbonate ions. Bone also contributes to buffering changes in hydrogen ion concentration. Generally, approximately 90% of bone mass is gained during childhood and adolescence in humans. Therefore, optimizing bone growth early in life is crucial for preventing fractures and osteoporosis later in life.

In children, malnutrition has been a leading cause of short stature and growth attenuation, which ultimately reflects on defective growth of long bones. In the 2021 World Bank joint report from UNICEF-WHO, 22% of all children under the age of 5 worldwide suffered linear growth restriction due to chronic malnutrition. In developing countries, that proportion increased to about 35%. Failure to grow healthy bone tissue during childhood impacts stature and can also cause serious problems later in life. The higher the bone mass acquired before adulthood, around the age of 20-25, the better prognosis for good bone health as bone mass declines with age throughout adulthood. An improvement in peak bone mass before adulthood can reduce the risk of osteoporotic fractures in adulthood.

Bone undergoes a constant and continuous process of remodeling where old bone is replaced by new bone. In the average human, approximately 5-10% of bone is renewed per year. This bone remodeling process occurs throughout life and is necessary for skeleton adaptation for mechanical use, promoting fracture healing and maintaining calcium homeostasis. Calcium homeostasis largely reflects the balance between osteoblast activity (or bone formation) and osteoclast activity (bone resorption).

Osteoclasts are responsible for bone resorption. They are terminally differentiated multinucleate cells that originate from mononuclear cells of the hematopoietic stem cell lineage. On the contrary, osteoblasts synthesize bone matrix through the deposition of organic matrix and the resulting mineralization. Osteoblasts are derived from pluripotent bone marrow mesenchymal stem cells (MSCs). MSCs can differentiate into different tissue-specific cells including osteoblasts, chondrocytes, and adipocytes. The commitment of MSCs to differentiate into osteoblasts requires the expression of specific genes.

In humans, longitudinal bone growth occurs rapidly in fetal life and early childhood, and then progressively ceases during adolescence. The elongation process of longitudinal bone growth, or endochondral ossification, is achieved by the activity of specialized cartilage structures known as growth plates located at the distal and proximal ends of long bones and vertebrae.

Growth plates are divided into five zones: resting, proliferative, hypertrophic cartilage, calcified cartilage, and ossification zones. The resting zone is located at the top of the growth plate, farthest from the ossification zone. The resting zone is composed of chondrocytes embedded within a cartilaginous matrix. This zone is crucial for maintaining the structure of the growth plate and may also be referred to as the “stem cell” zone. Chondrocytes within the resting zone undergo slow division to feed the adjacent proliferative zone. The proliferative zone organizes chondrocytes into vertical columns, where the chondrocytes undergo rapid mitotic divisions. Once the chondrocytes begin undergoing these divisions, they begin to enlarge rapidly and modify their extracellular matrix. This leads to the hypertrophic cartilage zone. Within the hypertrophic cartilage zone, the extracellular matrix is resorbed and reduced to thin septa between enlarged chondrocytes. In the adjacent calcified cartilage zone, hypertrophic chondrocytes undergo apoptosis and the thin septa become calcified. Lastly, bone tissue appears in the ossification zone. Cavities left by dying chondrocytes are invaded by osteoprogenitor cells that further differentiate into active osteoblasts. The osteoblasts are critical for bone formation as they deposit bone matrix over the calcified cartilage matrix.

Osteoblasts are not only important for longitudinal bone growth, but they are also important for appositional bone growth, or bone remodeling. Appositional bone growth is a process that occurs at the diaphysis of long bones. This growth changes the bone shape and structure while increasing the width of the bone. Appositional bone growth occurs while bones are increasing in length, but also continues after adolescence, i.e. even after the age of about 21 when growth plates have closed and longitudinal growth has ceased. The mechanism of appositional bone growth depends on a balance of osteoblast and osteoclast activity, as osteoblasts add bone matrix to external bone surfaces and osteoclasts remove bone from internal bone surfaces of the diaphysis.

Longitudinal bone growth and remodeling are complex processes where an equilibrium between bone formation and resorption is necessary for success. Although overall bone health can be actively promoted through adequate nutrition and exercise, there are many factors that can threaten bone growth and overall skeletal health. Such factors include malnutrition, malabsorption, vitamin insufficiency (primarily vitamins K, D and B), zinc and calcium deficiency, use of certain medications such as chemotherapeutic agents, long-term antibiotic or glucocorticoid use, diseases such as type 1 diabetes mellitus, type 2 diabetes mellitus, and osteoporosis, and abnormal hormonal balance during aging (including during and after menopause in women). Many of these factors can impair osteoblast differentiation and activity which leads to a disruption in normal bone growth and remodeling.

Osteoporosis is an age-related pathological condition characterized by a decrease in bone mineral density and bone mass. Approximately 1 in 3 women, and 1 in 5 men, over the age of 50 experience osteoporotic fractures. Age-related skeletal frailty such as that seen in osteoporosis can be attributed to impaired bone formation due to an insufficient amount of osteoblasts. This defective osteoblast number in the aging skeleton can be at least partly attributed to defective differentiation of progenitor cells or diversion of these progenitor cells towards adipocyte lineage. As such, developments aimed at enhancing osteoblast differentiation and activity are desirable to improve bone homeostasis and overall skeletal health in both adult and pediatric populations.

Previous treatments to combat disruptions to normal bone growth and remodeling have included supplements of calcium and vitamins D and K to restore bone homeostasis and prevent the onset and progression of bone diseases such as osteoporosis. However, many factors still impact the balance of bone homeostasis.

In view of the above, there is an urgent need to develop new and accessible technologies that can conveniently provide bone health improvements in vivo in human and animal subjects.

### SUMMARY OF THE INVENTION

It is therefore an object of the invention to provide a convenient means for improving bone health in an individual.

In one embodiment, the invention is directed to a method for improving bone formation in an individual comprising administering a bovine milk exosome-enriched product and vitamin K2 to the individual.

In another embodiment, the invention is directed to a method for reducing a risk of bone fracture or strengthening bone in an individual comprising administering a bovine milk exosome-enriched product and vitamin K2 to the individual.

In another embodiment, the invention is directed to a method for preventing or delaying onset or development of osteoporosis in an individual comprising administering a bovine milk exosome-enriched product and vitamin K2 to the individual.

The administration of a bovine milk exosome-enriched product and vitamin K2 provides a convenient means for improving bone health and development. These and additional objects and advantages of the invention will be more fully apparent in view of the following detailed description.

### BRIEF DESCRIPTION OF THE DRAWINGS

The drawings are illustrative of certain embodiments of the invention and are exemplary in nature and are not intended to limit the invention defined by the claims, wherein:

FIG. 1A illustrates osteoblast differentiation molecular marker Runx2 protein expression levels resulting from treatment with different effectors, as described in Example 2.

FIG. 1B illustrates osteoblast differentiation molecular marker LC3 protein expression levels resulting from treatment of different effectors, as described in Example 2.

FIG. 2A illustrates Western blots of osteoblast differentiation molecular marker Runx2 resulting from treatment with different effectors, as described in Example 2.

FIG. 2B illustrates Western blots of osteoblast differentiation molecular marker LC3 resulting from treatment with different effectors, as described in Example 2.

FIG. 3A illustrates a micrograph from an Alizarin Red S (ARS) extracellular mineralization assay of a control group of cells showing calcium deposits after 12 days of incubation, as described in Example 3.

FIG. 3B illustrates a micrograph from an ARS extracellular mineralization assay of an exosome-treated group of cells showing calcium deposits after 12 days of incubation, as described in Example 3.

FIG. 3C illustrates a micrograph from an ARS extracellular mineralization assay of a vitamin K2-treated group of cells showing calcium deposits after 12 days of incubation, as described in Example 3.

FIG. 3D illustrates a micrograph from an ARS extracellular mineralization assay of a vitamin K2+exosome-treated group of cells showing calcium deposits after 12 days of incubation, as described in Example 3.

### DETAILED DESCRIPTION

Specific embodiments of the invention are described herein. The invention can, however, be embodied in different forms and should not be construed as limited to the embodiments set forth herein. Rather, these embodiments are provided to illustrate more specific features of certain embodiments of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive of additional elements or steps, in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

All ranges and parameters, including but not limited to percentages, parts, and ratios disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

All percentages are percentages by weight unless otherwise indicated.

The term “an exosome-enriched product” as used herein, unless otherwise specified, refers to a product comprising bovine milk-derived exosomes in which exosomes have been substantially separated from other bovine milk components such as lipids, cells, and debris, and are concentrated in an amount higher than that found in bovine milk. Exosomes are small, extracellular vesicles that account for a minor percentage of milk's total content. In specific embodiments, the exosome-enriched product is administered in the form of an exosome-enriched liquid or an exosome-enriched powder. In certain embodiments, the exosome-enriched product also contains co-isolated milk solids. Bovine milk exosomes are extracellular membrane vesicles of approximately 20-200 nm in diameter. These nanosized structures contain several bioactive agents, including, but not limited to, enzymatic and non-enzymatic proteins (e.g., CD9, CD63, MHC-class II, lactadherin, TSG101 and Hsc70), nucleic acids (including high amounts of microRNA (miRNA) and messenger RNA (mRNA)) and lipids (e.g., phosphatidylethanolamine, phosphatidylserine, phosphatidylcholine and sphingomyelin).

The term “intact exosomes” as used herein, unless otherwise specified, refers to exosomes in which the vesicle membrane is not ruptured and/or otherwise degraded and the endogenous cargo, i.e., the bioactive agents, therapeutics (e.g. miRNA), and/or other biomolecules which are inherently present in bovine milk exosomes, are retained therein in active form.

In embodiments of the invention, the bovine milk exosome-enriched product comprises intact bovine milk exosomes. In a specific embodiment, at least about 50 wt % of the exosomes in the bovine milk exosome-enriched product are intact. In another specific embodiment, at least about 55, 60, 70, 75, 80, 85, 90, or 95 wt % of the exosomes in the bovine milk exosome-enriched product are intact.

Bovine milk exosomes can be isolated from a milk whey fraction or from other dairy streams, such as a cheese whey fraction. They can be isolated by various physical (e.g., ultracentrifugation at increasing speeds, membrane ultrafiltration and/or size exclusion chromatography) and/or chemical methods (e.g., the use of polymers to precipitate bovine milk exosomes by an incubation step). Remarkably, most of these procedures tend to co-purify exosomes and other dairy constituents (i.e., caseins and other whey protein). The isolation process yields a fraction enriched in bovine milk exosomes that may then undergo further processing, such as freeze-drying or spray-drying, to produce a powder containing bovine milk exosomes for further applications.

In a specific embodiment of the invention, the bovine milk exosome-enriched product comprises at least 0.001 wt % exosomes. In another specific embodiment, the bovine milk exosome-enriched product comprises at least about 0.001, 0.01, 1, 5, 10, 15, 20, 25, 30, 35, 40, 45, or 50 wt % exosomes. In additional specific embodiments of the invention, the bovine milk exosome-enriched product comprises at least 10 wt % exosomes. In further embodiments, the bovine milk exosome-enriched product comprises at least about 10⁸ exosomes per gram as measured by a nanotracking procedure. Briefly, nanoparticle tracking analysis (NTA) can be used to determine exosome diameter and concentration. The principle of NTA is based on the characteristic movement of nanosized particles in solution according to the Brownian motion. The trajectory of the particles in a defined volume is recorded by a camera that is used to capture the scatter light upon illumination of the particles with a laser. The Stokes-Einstein equation is used to determine the size of each tracked particle. In addition to particle size, this technique also allows determination of particle concentration.

In a more specific embodiment, the bovine milk exosome-enriched product employed in the present invention comprises from about 10⁸ to about 10¹⁵ exosomes per gram of the exosome-enriched product. In yet a more specific embodiment, the exosome-enriched product comprises from about 10⁹ to about 10¹³ exosomes per gram of the exosome-enriched product. In another specific embodiment, the exosome-enriched product contains at least about a three-fold increase in the number of exosomes, as compared to a raw whey-containing bovine milk fraction. In a more specific embodiment, the exosome-enriched product contains a 3-fold to 50-fold increase in the number of exosomes, as compared to a raw whey-containing bovine milk fraction, for example cheese whey.

The term “vitamin K2” as used herein, unless otherwise specified, refers to a fat-soluble vitamin also known as menaquinone having a variable side chain length of four to 15 isoprene units and referred to as MK-n, where n denotes the number of isoprenoid units. Typically, vitamin K2 has an average of 7 isoprenoid units and is referred to as MK-7. Vitamin K2 can be obtained commercially through different extraction and purification processes.

The terms “mesenchymal stem cells” or “MSCs” refer to pluripotent bone marrow cells that can differentiate into different tissue-specific cells such as osteoblasts, chondrocytes and adipocytes.

Runx2 (Runt-related transcription factor 2) is an early osteoblast differentiation marker. Runx2 can induce expression of bone matrix protein genes and mineralization in osteoblastic cells in vitro. Certain factors or diseases such as long-term glucocorticoid use can increase the incidence of osteoporosis and suppress osteogenic differentiation of MSCs by, for example, antagonizing Runx2. Runx2 is considered a master osteogenic transcription factor, being essential for the expression of osteogenic differentiation genes. In embodiments of the invention, activity of differentiated osteoblasts are evaluated through Runx2 expression levels.

LC3 (microtubule associated protein 1 light chain 3-α) is a late osteoblast differentiation marker. LC3 is a protein required for MC3T3-E1 osteoblast differentiation and mineralization through autophagy induction. A lack of autophagy in osteoblasts can decrease mineralization activity. In embodiments of the invention, differentiated osteoblasts are evaluated through LC3 expression levels.

Alizarin Red S (ARS) assay is an anthraquinone dye used to visualize and evaluate extracellular calcium deposits in cell culture. In embodiments of the invention, mineralization activity of differentiated osteoblasts are evaluated through ARS assay.

The term “nutritional composition” as used herein, unless otherwise specified, refers to nutritional liquids and nutritional powders, the latter of which may be reconstituted or otherwise mixed with a liquid in order to form a nutritional liquid, and are suitable for oral consumption by a human. Nutritional liquids may be prepared in ready-to-drink (RTD) form or may be reconstituted from powder as described.

In embodiments of the invention, the vitamin K2 and exosomes of the bovine milk exosome-enriched product can be administered separately or in combination. Combinations may be prepared by stirring, mixing, shaking, or otherwise combining the vitamin K2 and exosomes, with care being exercised to substantially maintain the exosomes in intact form.

In a specific embodiment of the invention, vitamin K2 is loaded on exosomes of the bovine milk exosome-enriched product. In other specific embodiments, the vitamin K2 is a component separate from the exosomes, i.e., the vitamin K2 is not loaded on exosomes of the bovine milk exosome-enriched product.

In one embodiment of the invention, the single stirring step occurs by vortex for about 60 minutes, or about 40-80 minutes, or about 50-70 minutes. In specific embodiments of the invention, the vortex occurs at 500 rpm or more, or about 500-1300 rpm, or about 600-1200 rpm, or about 1000-1200 rpm, or about 1200 rpm.

In specific embodiments of the invention, a bovine milk exosome-enriched product and vitamin K2 are administered to a subject, where the subject may be a human adult, older adult, or pediatric individual.

The term “pediatric subject” as used herein, unless otherwise specified, refers to an infant, child or adolescent individual up to the age of about 20 years old. In specific embodiments of the invention, the pediatric subject is a child at or under the age of about 18 years old, or at or under the age of about 15 years old, or at or under the age of about 10 years old, or at or under the age of about 5 years old, or at or under the age of about 1 year old, or at or under the age of about 6 months old, or at or under the age of about 3 months old.

The term “adolescent” as used herein, unless otherwise specified, refers to a pediatric subject at the age of about 10 years to 20 years old, which normally corresponds with the onset of physiologically normal puberty and ends when an adult identity and behavior are accepted. A pediatric subject under the age of about 10 years old corresponds to an individual in “childhood.”

In specific embodiments of the invention, a bovine milk exosome-enriched product and vitamin K2 are administered to a pediatric subject.

The term “adult subject” as used herein, refers to an individual at least the age of about 20 years old.

The term “older adult” as used herein, refers to an adult subject at least the age of about 50 years old.

In certain embodiments of the invention, a bovine milk exosome-enriched product and vitamin K2 are administered to an adult subject. In embodiments of the invention, the adult subject is at least about 30 years old, or at least about 40 years old, or at least about 50 years old, or at least about 60 years old, or at least about 70 years old, or is about 40-80 years old, or is about 50-70 years old.

In specific embodiments of the invention, the adult subject comprises an adult of at least about 40 years old, or at least about 50 years old, or at least about 60 years old, or at least about 65 years old, or about 50-80 years old, or about 60-70 years old, or about 63-64 years old.

The term “bone development” as used herein, refers to the continuous process where old bone is replaced by new bone by maintaining bone homeostasis with a balance between bone formation (via osteoblast activity) and bone resorption (via osteoclast activity). Osteoblasts are specialized cells derived from mesenchymal cells that synthesize bone matrix. Osteoclasts are cells that degrade bone to contribute to bone remodeling.

The term “bone formation” as used herein, unless otherwise specified, refers to the portion of bone development where osteoblast activity is stimulated. Osteoblasts add bone matrix to external bone surfaces through the deposition of organic matrix and its mineralization. Bone formation may refer to both longitudinal bone growth as well as appositional bone growth. Most bone formation, in the normal course of growing mammals, occurs during childhood and adolescence. However, bone formation continues to facilitate bone repair, for example, of a fracture, throughout life due to the same actions of osteoblasts. As such, the term bone formation may also include bone repair.

The term “bone resorption” as used herein, unless otherwise specified, refers to the portion of bone development where osteoclast activity is stimulated. Osteoclasts remove bone from the internal surfaces of the diaphysis of long bones by breaking down bone matrix through phagocytosis.

The term “bone fracture” as used herein, unless otherwise specified, refers to a partial or complete break in a bone. A bone fracture may also include a crack in a bone.

The term “osteoporosis” as used herein, unless otherwise specified, refers to a condition where bones become weak and/or brittle. In osteoporosis, bone homeostasis is not maintained as osteoblast activity to generate new bone does not keep up with osteoclast activity to remove old bone.

In one embodiment, the invention is directed to a method for improving bone formation in an individual comprising administering a bovine milk exosome-enriched product and vitamin K2 to the individual.

In another embodiment, the invention is directed to a method for reducing a risk of bone fracture or strengthening bone in an individual comprising administering a bovine milk exosome-enriched product and vitamin K2 to the individual.

In another embodiment, the invention is directed to a method for preventing or delaying onset or development of osteoporosis in an individual comprising administering a bovine milk exosome-enriched product and vitamin K2 to the individual.

All the methods as disclosed herein can also be considered as use of a bovine milk exosome-enriched product and vitamin K2 for the described improvements in bone health.

In specific embodiments, the invention is directed to a medicament for use in improving bone formation in an individual and comprises a bovine milk exosome-enriched product and vitamin K2.

In another embodiment, the invention is directed to a medicament for use in reducing a risk of bone fracture or strengthening bone in an individual and comprises a bovine milk exosome-enriched product and vitamin K2.

In yet another embodiment, the invention is directed to a medicament for use in preventing or delaying onset or development of osteoporosis in an individual and comprises a bovine milk exosome-enriched product and vitamin K2.

In a specific embodiment of the invention, the dosage of the exosome-enriched product is from about 0.01 to about 30 g per day. More specifically, the dosage of the exosome-enriched product may be from about 0.1 to about 30 g, from about 0.1 to about 15 g, or from about 1 to about 15 g per day. In certain embodiments of the invention, the dosage of the exosome-enriched product is administered in accordance with the characteristics of the exosome-enriched product as described above, specifically, wherein the bovine milk exosome-enriched product comprises from about 10⁸ to about 10¹⁵ exosomes per gram of the exosome-enriched product.

In a specific embodiment of the invention, the dosage of the vitamin K2 follows the recommended daily allowance (RDA) of vitamin K. For example, the RDA of vitamin K is 120 micrograms (mcg) of vitamin K for adult males per day, 90 mcg for adult females per day, 30 mcg for children aged 1-3 years old per day, 55 mcg for children aged 4-8 years old per day, 60 mcg for children aged 9-13 years old per day, and 75 mcg for adolescents aged 14-18 years old per day. In one embodiment, vitamin K2 is administered at a dose of about 0.001 to about 0.3 mg, or about 0.03 to about 0.12 mg per day. In yet another embodiment, vitamin K2 is administered at a dose of about 0.03 mg, 0.055 mg, 0.06 mg, 0.075 mg, 0.09 mg, or 0.12 mg per day.

In specific embodiments, the bovine milk exosome-enriched product and vitamin K2 are administered to an individual at least once daily. In specific embodiments, the bovine milk exosome-enriched product and vitamin K2 are administered to an individual about 1-2 times per day.

In specific embodiments, the bovine milk exosome-enriched product and vitamin K2 are administered to the individual at least once daily, or from about once or twice daily for a period of at least 3 consecutive days, at least one week, at least 12 consecutive days, at least two weeks, at least three weeks, or at least four weeks.

In some aspects of the invention, vitamin K2 and the bovine milk exosome-enriched product are included in a nutritional composition. The nutritional composition further includes protein, carbohydrate, and/or fat.

The vitamin K2 is included in the nutritional composition in an amount sufficient to provide the desired vitamin K2 content, for example, the recommended daily allowance. Therefore, in one embodiment, the nutritional composition contains vitamin K2 in an amount sufficient to provide at least about 30 micrograms (0.03 mg) vitamin K2 per serving, for example, in an 8 oz/237 ml serving.

In other specific embodiments, the nutritional composition comprises at least about 0.0000004 wt %, or at least about 0.000025 wt %, or at least about 0.00005 wt %, or at least about 0.0001 wt %, or at least about 0.0002 wt % of vitamin K2, based on the weight of the nutritional composition. In a specific embodiment, the nutritional composition comprises from about 0.0000004 wt % to about 0.00005 wt %, or from about 0.000025 to about 0.0002 wt % of vitamin K2, based on the weight of the nutritional composition.

In other specific embodiments, the nutritional composition comprises about 0.001 to about 30 wt %, about 0.001 to about 10 wt %, about 0.001 to about 5 wt %, about 0.001 to about 1 wt %, about 0.01 to about 10 wt %, about 0.01 to about 5 wt %, about 0.01 to about 1 wt %, about 0.1 to about 10 wt %, about 0.1 to about 5 wt %, about 0.1 to about 1 wt %, about 1 to about 10 wt %, or about 1 to about 5 wt % of the exosome-enriched product, based on the weight of the nutritional composition. In a specific embodiment, the nutritional composition comprises from about 0.001 to about 30 wt % of exosomes loaded with vitamin K2, based on the weight of the nutritional composition.

In another specific embodiment, the nutritional composition comprises one or more carbohydrates comprising fiber, including, but not limited to oat fiber, soy fiber, and/or corn fiber, human milk oligosaccharides (HMOs), maltodextrin, corn maltodextrin, organic corn, corn syrup, sucralose, cellulose gel, cellulose gum, gellan gum, inositol, carrageenan, fructooligosaccharides, hydrolyzed starch, glucose polymers, corn syrup solids, rice-derived carbohydrates, sucrose, glucose, lactose, honey, sugar alcohols, isomaltulose, sucromalt, pullulan, potato starch, galactooligosaccharides, gum arable, sodium carboxymethylcellulose, methylcellulose, guar gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, psyllium, inulin, cornstarch, or combinations of two or more thereof.

The nutritional composition may comprise carbohydrate in an amount from about 5 wt % to about 75 wt % of the nutritional composition. More specifically, the carbohydrate may be present in an amount from about 5 wt % to about 70 wt % of the nutritional composition, including about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, about 40 wt % to about 70 wt %, or about 15 wt % to about 25 wt %, of the nutritional composition.

In another specific embodiment, the nutritional composition comprises one or more fats comprising coconut oil, fractionated coconut oil, soy oil, soy lecithin, corn oil, safflower oil, high oleic sunflower oil, palm olein, canola oil monoglycerides, lecithin, medium chain triglycerides, linoleic acid, alpha-linolenic acid, arachidonic acid, eicosapentaenoic acid, docosahexaenoic acid, olive oil, high gamma linolenic (GLA) safflower oil, palm oil, palm kernel oil, canola oil, marine oils, fish oils, algal oils, borage oil, cottonseed oil, fungal oils, interesterified oils, transesterified oils, structured lipids, or combinations of two or more thereof.

The nutritional composition may comprise fat in an amount of from about 0.5 wt % to about 30 wt % of the nutritional composition. More specifically, the fat may be present in an amount from about 0.5 wt % to about 10 wt %, about 1 wt % to about 30 wt % of the nutritional composition, including about 1 wt % to about 20 wt %, about 1 wt % to about 15 wt %, about 1 wt % to about 10 wt %, about 1 wt % to about 5 wt %, about 3 wt % to about 30 wt %, about 5 wt % to about 30 wt %, about 5 wt % to about 25 wt %, about 5 wt % to about 20 wt %, about 5 wt % to about 10 wt %, or about 10 wt % to about 20 wt % of the nutritional composition.

In another specific embodiment, the nutritional composition comprises one or more proteins comprising whey protein concentrate, whey protein isolate, whey protein hydrolysate, acid casein, sodium caseinate, calcium caseinate, potassium caseinate, casein hydrolysate, milk protein concentrate, organic milk protein concentrate, milk protein isolate, milk protein hydrolysate, nonfat dry milk, condensed skim milk, soy protein concentrate, isolated soy protein, soy protein hydrolysate, pea protein concentrate, pea protein isolate, pea protein hydrolysate, collagen protein, collagen protein isolate, L-Carnitine, L-Lysine, taurine, lutein, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, guinea proteins, amaranth proteins, chia proteins, hemp proteins, flax seed proteins, earthworm protein, insect protein, one or more amino acids and/or metabolites thereof, or combinations of two or more thereof.

The one or more amino acids, which may be described as free amino acids, can be any amino acid known for use in nutritional products. The amino acids may be naturally occurring or synthetic amino acids. In a specific embodiment, the one or more amino acids and/or metabolites thereof comprise one or more branched chain amino acids or metabolites thereof. Examples of branched chain amino acids include arginine, glutamine leucine, isoleucine, and valine. In another specific embodiment, the one or more branched chain amino acids or metabolites thereof comprise alpha-hydroxy-isocaproic acid (HICA, also known as leuic acid), keto isocaproate (KIC), β-hydroxy-β-methylbutyrate (HMB), and combinations of two or more thereof.

The nutritional composition may comprise protein in an amount from about 1 wt % to about 30 wt % of the nutritional composition. More specifically, the protein may be present in an amount from about 1 wt % to about 25 wt % of the nutritional composition, including about 1 wt % to about 20 wt %, about 2 wt % to about 20 wt %, about 1 wt % to about 15 wt %, about 1 wt % to about 10 wt %, about 5 wt % to about 10 wt %, about 10 wt % to about 25 wt %, or about 10 wt % to about 20 wt % of the nutritional composition. Even more specifically, the protein comprises from about 1 wt % to about 5 wt % of the nutritional composition, or from about 20 wt % to about 30 wt % of the nutritional composition.

The concentration and relative amounts of the sources of protein, carbohydrate, and fat in the exemplary nutritional compositions can vary considerably depending upon, for example, the specific dietary needs of the intended user. In a specific embodiment, the nutritional composition comprises a source of protein in an amount of about 2 wt % to about 20 wt %, a source of carbohydrate in an amount of about 5 wt % to about 30 wt %, and a source of fat in an amount of about 0.5 wt % to about 10 wt %, based on the weight of the nutritional composition, and, more specifically, such composition is in liquid form. In another specific embodiment, the nutritional composition comprises a source of protein in an amount of about 10 wt % to about 25 wt %, a source of carbohydrate in an amount of about 40 wt % to about 70 wt %, and a source of fat in an amount of about 5 wt % to about 20 wt %, based on the weight of the nutritional composition, and, more specifically, such composition is in powder form.

In one embodiment, the nutritional composition is a liquid nutritional composition and comprises from about 1 to about 15 wt % of protein, from about 0.5 to about 10 wt % fat, and from about 5 to about 30 wt % carbohydrate, based on the weight of the nutritional composition.

In another embodiment, the nutritional composition is a powder nutritional composition and comprises from about 10 to about 30 wt % of protein, from about 5 to about 15 wt % fat, and from about 30 wt % to about 65 wt % carbohydrate, based on the weight of the nutritional composition.

The nutritional composition may also comprise one or more components to modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth. The nutritional compositions may also include additional vitamins and minerals, for example in amounts complying with the recommended daily allowances.

In specific embodiments, the nutritional composition has a neutral pH, i.e., a pH of from about 6 to 8 or, more specifically, from about 6 to 7.5. In more specific embodiments, the nutritional composition has a pH of from about 6.5 to 7.2 or, more specifically, from about 6.8 to 7.1.

The nutritional composition may be formed using any techniques known in the art. In one embodiment, the nutritional composition may be formed by (a) preparing an aqueous solution comprising protein and carbohydrate; (b) preparing an oil blend comprising fat and oil-soluble components; and (c) mixing together the aqueous solution and the oil blend to form an emulsified liquid nutritional composition. The bovine milk exosome-enriched product and vitamin K2 may be added at any time as desired in the process, for example, to the aqueous solution or to the emulsified blend. The bovine milk-derived exosome-enriched product and vitamin K2 may be dry blended in powder form with one or more dry ingredients, for example, for combined addition to a liquid composition or if a powdered nutritional product is desirable.

In a specific embodiment, the nutritional composition is administered in the form of a powder. In another specific embodiment, the nutritional composition is administered in the form of a liquid. The nutritional composition can be administered to an individual in either form. When the nutritional composition is a powder, for example, a serving size is from about 40 g to about 60 g, such as 45 g, or 48.6 g, or 50 g, to be administered as a powder or to be reconstituted in from about 1 ml to about 500 ml of liquid.

When the nutritional composition is in the form of a liquid, for example, reconstituted from a powder or manufactured as a ready-to-drink product, a serving ranges from about 1 ml to about 500 ml, including from about 110 ml to about 500 ml, from about 110 ml to about 417 ml, from about 120 ml to about 500 ml, from about 120 ml to about 417 ml, from about 177 ml to about 417 ml, from about 207 ml to about 296 ml, from about 230 m to about 245 ml, from about 110 ml to about 237 ml, from about 120 ml to about 245 ml, from about 110 ml to about 150 ml, and from about 120 ml to about 150 ml. In specific embodiments, the serving is about 1 ml, or about 100 ml, or about 225 ml, or about 237 ml, or about 500 ml.

As indicated above, the present invention provides methods for improving bone formation, reducing a risk of bone fracture, strengthening bone, and preventing or delaying onset or development of osteoporosis in individuals comprising administering a bovine milk exosome-enriched product and vitamin K2.

The following Examples demonstrate various embodiments of the invention.

### EXAMPLES

### Example 1: Method for Preparing an Exosome-Enriched Product

This example describes a method of preparing an exosome-enriched product from cheese whey. The cheese whey was provided by adding rennet enzyme to bovine milk, resulting in enzymatic coagulation of casein and production of sweet cheese whey.

An exosome-enriched product containing from about 10⁸ to about 10¹⁵ intact bovine milk-derived exosomes per gram of the exosome-enriched product was prepared by cascade membrane filtration. First, 1,000 L of the sweet cheese whey was processed using tandem multiple ceramic filtration steps. The first microfiltration (MF) step employed a membrane with a molecular weight cut off of 1.4 μm, which yielded a first retentate R1 and a first permeate P1. The first permeate P1 was then subjected to an ultrafiltration step (UF) with a molecular weight cut off of 0.14 μm, which yielded a second retentate R2 and a second permeate P2. About 5 volumes of water was added to one volume of the second retentate R2, and the diluted retentate was then passed through the 0.14 μm UF membrane again to remove at least part of the lactose and minerals. The resulting retentate R3 was then combined with an equal volume of water and diafiltered using a 10 kDa membrane to produce a fourth retentate R4. The retentate R4 was diluted with a volume of water five times that of the fourth retentate R4 and diafiltered a second time using the 10 kDa membrane to yield a concentrated retentate, R5. The lactose-free exosome-enriched product R5 was pasteurized at 72° C. for 15 seconds to ensure microbiological stability in order to yield a pasteurized exosome-enriched product R6. The pasteurized exosome-enriched product R6 was subjected to evaporation at about 65° C. to increase the solids content up to 17-18% and then spray-dried at 185° C./85° C. to obtain an exosome-enriched spray-dried product.

### Example 2: In Vitro Cell Model Monitoring Osteoblast Differentiation Markers

This example describes the surprising effect of a combination of bovine milk exosomes and vitamin K2 on osteoblast differentiation and activity using murine MC3T3-E1 cells. MC3T3-E1 cells are immature osteoblasts and widely used as models of immature osteoprogenitor cell lines with the ability to differentiate into osteoblasts.

The cells used were evaluated for stimulated osteoblast activity via extracellular matrix mineralization and for the induced expression of osteoblast differentiation markers Runx2 and LC3.

To begin the assay, MC3T3-E1 pre-osteoblast cells were grown in MEM (minimum essential medium) culture medium supplemented with 10% v/v FBS (fetal bovine serum), 4 nM glutamine, 100 units/ml penicillin and 0.1 mg/ml streptomycin in a 5% CO2 and an atmosphere with 95% humidity. For osteogenic differentiation, these pre-osteoblast cells were seeded in 24-well plates at a density of 3×10⁴ cells/well in complete medium for 1 day. The medium was then replaced by MEM containing 10% v/v FBS, 4 nM glutamine, 100 units/ml penicillin, 0.1 mg/ml streptomycin, 10 nM β-glycerophosphate, and 50 μg/ml L-ascorbic acid.

The expression of differentiation markers Runx2 and LC3 was analyzed by Western blot after treatment with different effectors in differentiation medium, namely, bovine milk exosomes and/or vitamin K2.

Specifically, three groups of MC3T3-E1 pre-osteoblast cells were treated. Group 1 was treated with 1 μM vitamin K2. Group 2 was treated with 15 μg/ml bovine milk exosomes. Group 3 was treated with a combination of 1 μM vitamin K2 and 15 μg/ml bovine milk exosomes. Group 4 was included as an untreated control group.

After treatment with the effectors, the cells were lysed with an Radioimmunoprecipitation assay (RIPA) buffer (supplemented with phosphatase and protease inhibitors), 10 mM sodium fluoride, 10 mM sodium pyrophosphate, 1 mM sodium orthovanadate, 1 mM egtazic acid (EGTA), and 1 mM phenylmethylsulfonyl fluoride (PMSF). Total protein was denatured by heating the cells at 95° C. for 5 minutes and 20 μg of protein was ran on a 10% acrylamide sodium dodecyl sulfate-polyacrylamide gel electrophoresis (SDS-PAGE) gel. The proteins were then subsequently transferred to nitrocellulose membranes and immunoblotted with specific antibodies (Runx2-Ref: 1L7F, cell signaling, and LC3-Ref: 4108, cell signaling). Glyceraldehyde 3-phosphate dehydrogenase (GAPDH) was used as a normalizer.

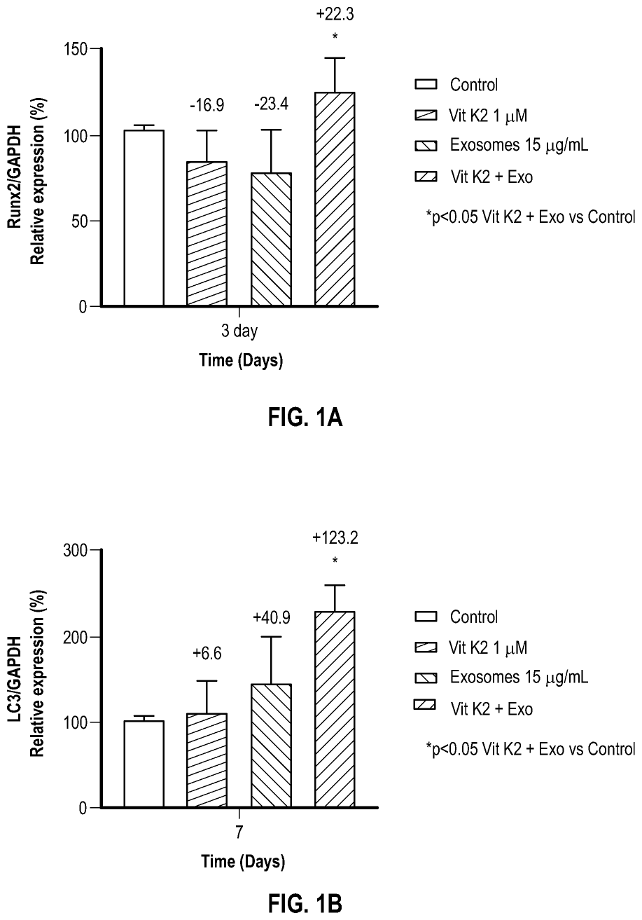

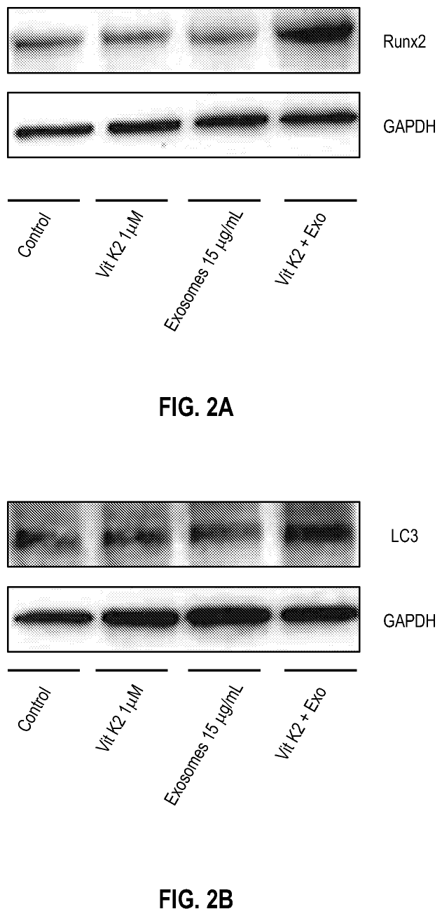

FIG. 1A shows the resulting expression of Runx2 protein levels after 3 days of incubation treatment for all four groups. As shown, Group 3 where the MC3T3-E1 pre-osteoblast cells were incubated with a combination of vitamin K2 and bovine milk exosomes, showed a statistically significant increase of Runx2 osteogenic differentiation over Groups 1 and 2, treated separately with vitamin K2 and bovine milk exosomes, respectively. Group 3 also showed an increase (22.3%) in Runx2 expression over the control Group 4. These results indicate the surprising effect that the combination of bovine milk exosomes and vitamin K2 have on Runx2 expression, over either of these effectors alone, as well as over the control group. These results are also shown in the Western blot of FIG. 2A.

FIG. 1B shows the resulting expression of LC3 protein levels after 7 days of incubation treatment for all four groups. As shown, Group 3 where the MC3T3-E1 pre-osteoblast cells were incubated with a combination of vitamin K2 and bovine milk exosomes, showed a statistically significant increase of LC3 osteogenic differentiation over Groups 1 and 2, treated separately with vitamin K2 and bovine milk exosomes, respectively. Group 3 also showed an increase (123.2%) in LC3 expression over the control Group 4. These results indicate the surprising effect that the combination of bovine milk exosomes and vitamin K2 also have on LC3 expression, over either of these effectors alone, as well as over the control group. These results are also shown in the Western blot of FIG. 2B.

### Example 3: In Vitro Cell Model Monitoring Calcium Deposits

In addition to analyzing Runx2 and LC3 molecular markers in Example 2, cell Groups 1-3 as described in Example 2 were treated in the presence of a differentiation medium for 12 days. Every 3 days the differentiation media with effectors was replaced with fresh differentiation media and effectors. Group 4 from Example 2 was also maintained as the control group.

After 12 days of incubation, supernatant was removed from all three treated groups and the cells were washed twice with PBS and fixed with 2% paraformaldehyde at room temperature for 20 minutes. Calcium nodes in all three groups were stained with Alizarin Red (2003999, MERCK) at 37° C. for 1 hour. The calcium nodes were then observed under a microscope and representative micrographs were taken of each group.

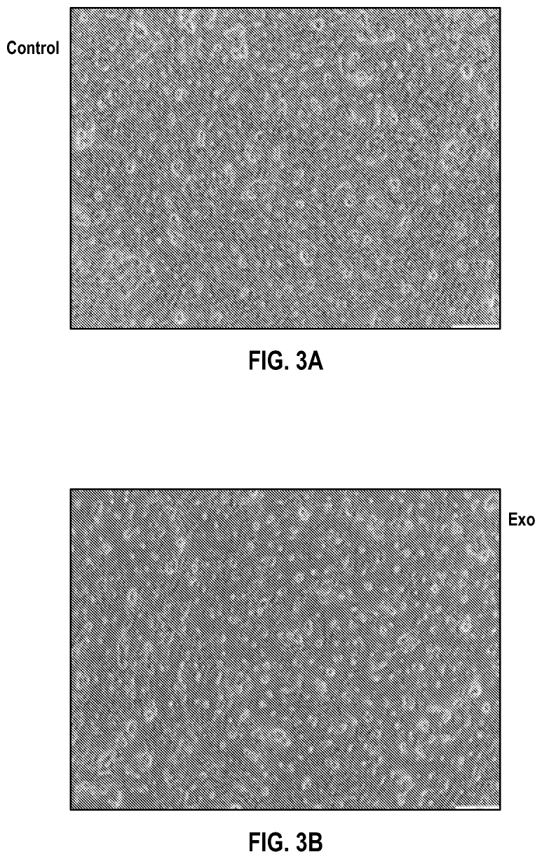

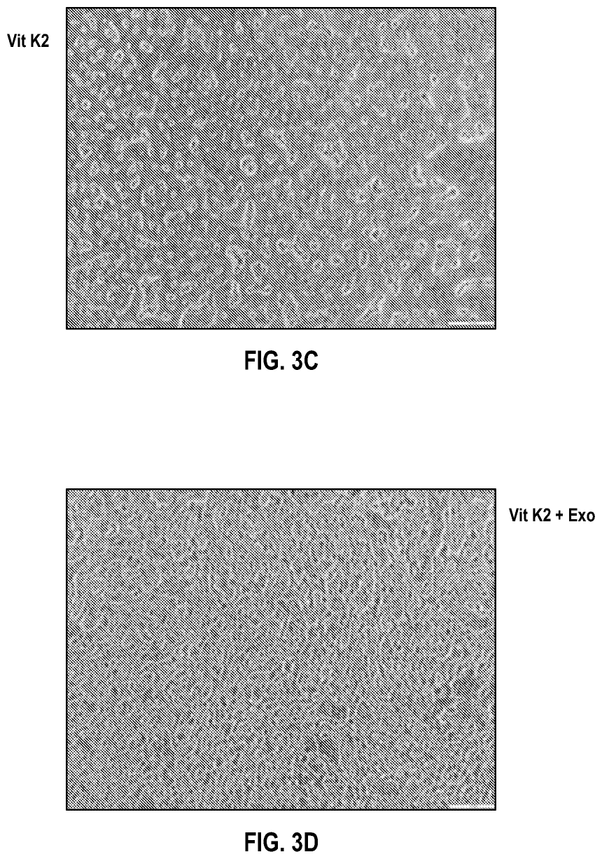

The resulting micrographs are shown in FIGS. 3A-3D. FIG. 3A shows the calcium deposits from control Group 4. FIG. 3B shows the calcium deposits from Group 2, treated with bovine milk exosomes. FIG. 3C shows the calcium deposits from Group 1, treated with vitamin K2. FIG. 3D shows the calcium deposits from Group 3, treated with both bovine milk exosomes and vitamin K2.

ARS assay allows for the visualization of calcium deposits produced by active osteoblasts. While all of FIGS. 3A-3D show abundant calcium deposits in the micrographs, FIG. 3D, which shows the results of Group 3 treated with both effectors vitamin K2 and bovine milk exosomes, surprisingly induced enhanced extracellular matrix mineralization with much more calcium deposits than the other three groups (treated Groups 1-2 and control Group 4). This shows that not only does the combination of vitamin K2 and bovine milk exosomes promote an unexpected level of osteoblast differentiation from the results of Example 2, but this combination also surprisingly increases osteoblast mineralization activity (based on increased calcium deposits).

The combination of the surprising results of Example 2 and Example 3 indicate that the combination of vitamin K2 and bovine milk exosomes represents an unexpected treatment to increase osteoblast differentiation and activity, and therefore provides for improved bone formation, reduced risk of bone fracture, and preventing or delaying onset or development of osteoporosis in an individual.

In summary, the present invention provides methods that increase osteoblast differentiation and activity for improved bone health by administering vitamin K2 and a bovine milk exosome-enriched product. Such methods are useful for efficient and/or large-scale production of various nutritional compositions, especially for growing pediatric individuals experiencing long bone growth, and for older adult individuals more prone to osteoporosis.

The specific embodiments and examples described herein are exemplary only and are not limiting to the invention defined in the claims. Additionally, while the present invention has been illustrated by the description of embodiments thereof, and while the embodiments have been described in considerable detail, such descriptions are not intended to restrict or in any way limit the scope of the appended claims to such detail. Additional advantages and modifications will readily appear to those skilled in the art. Therefore, the invention, in its broader aspects, is not limited to the specific details, the representative compositions and processes, or illustrative examples shown and described. Accordingly, departures may be made from such details without departing from the spirit or scope of the general inventive concept.

1. A method for improving bone formation in an individual comprising administering a bovine milk exosome-enriched product and vitamin K2 to the individual.

2. The method of claim 1, further comprising reducing a risk of bone fracture or strengthening bone in the individual.

3. (canceled)

4. The method according to claim 1, wherein the individual is a pediatric subject.

5.-6. (canceled)

7. The method according to claim 1, wherein the bovine milk exosome-enriched product is administered in the form of an exosome-enriched liquid nutritional composition.

8.-9. (canceled)

10. The method according to claim 1, wherein the bovine milk exosome-enriched product comprises from about 10⁸ to about 10¹⁵ exosomes per gram of the exosome-enriched product.

11. The method according to claim 1, wherein the bovine milk exosome-enriched product comprises intact bovine milk-derived exosomes.

12. The method of claim 11, wherein at least about 50 wt % of exosomes in the bovine milk exosome-enriched product are intact.

13. The method of claim 11, wherein at least about 90 wt % of the exosomes in the bovine milk exosome-enriched product are intact.

14. The method according to claim 1, wherein the exosome-enriched product is administered to the individual at a dose of about 0.01 to about 30 g per day.

15. The method according to claim 1, wherein vitamin K2 is administered to the individual at a dose of about 0.001 to about 0.3 mg per day.

16. The method according to claim 1, wherein the bovine milk exosome-enriched product and vitamin K2 are administered to the individual at least once daily.

17. The method according to claim 1, wherein the bovine milk exosome-enriched product and vitamin K2 are administered to the individual at least once daily for at least about 3 consecutive days.

18. The method according to claim 1, wherein the exosome-enriched product and vitamin K2 are administered to the individual in a nutritional composition comprising protein, carbohydrate, and/or fat.

19. The method according to claim 19, wherein the nutritional composition comprises from about 0.001 to about 30 wt % of the exosome-enriched product, based on the weight of the nutritional composition.

20. The method according to claim 19, wherein the nutritional composition comprises at least about 0.0000004 wt %, or at least about 0.000025 wt %, or at least about 0.00005 wt %, or at least about 0.0001 wt %, or at least about 0.0002 wt % of vitamin K2, based on the weight of the nutritional composition.

21. The method according to claim 20, wherein the nutritional composition is administered in the form of a liquid having a serving size ranging from 110 mL to 500 mL.

22. (canceled)

23. The method according to claim 18, wherein the nutritional composition comprises one or more proteins comprising whey protein concentrate, whey protein isolate, whey protein hydrolysate, acid casein, sodium caseinate, calcium caseinate, potassium caseinate, casein hydrolysate, milk protein concentrate, organic milk protein concentrate, milk protein isolate, milk protein hydrolysate, nonfat dry milk, condensed skim milk, soy protein concentrate, isolated soy protein, soy protein hydrolysate, pea protein concentrate, pea protein isolate, pea protein hydrolysate, collagen protein, collagen protein isolate, L-Carnitine, L-Lysine, taurine, lutein, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, guinea proteins, amaranth proteins, chia proteins, hemp proteins, flax seed proteins, earthworm protein, insect protein, one or more amino acids and/or metabolites thereof, or combinations of two or more thereof.

24. The method according to claim 18, wherein the nutritional composition comprises one or more fats comprising coconut oil, fractionated coconut oil, soy oil, soy lecithin, corn oil, safflower oil, high oleic sunflower oil, palm olein, canola oil monoglycerides, lecithin, medium chain triglycerides, linoleic acid, alpha-linolenic acid, arachidonic acid, eicosapentaenoic acid, docosahexaenoic acid, olive oil, high gamma linolenic (GLA) safflower oil, palm oil, palm kernel oil, canola oil, marine oils, fish oils, algal oils, borage oil, cottonseed oil, fungal oils, interesterified oils, transesterified oils, structured lipids, or combinations of two or more thereof.

25. The method according to claim 18, wherein the nutritional composition comprises one or more carbohydrates comprising fiber, human milk oligosaccharides (HMOs), maltodextrin, corn maltodextrin, organic corn, corn syrup, sucralose, cellulose gel, cellulose gum, gellan gum, inositol, carrageenan, fructooligosaccharides, hydrolyzed starch, glucose polymers, corn syrup solids, rice-derived carbohydrates, sucrose, glucose, lactose, honey, sugar alcohols, isomaltulose, sucromalt, pullulan, potato starch, galactooligosaccharides, gum arable, sodium carboxymethylcellulose, methylcellulose, guar gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, psyllium, inulin, cornstarch, or combinations of two or more thereof.
